(19) United States
(12) Patent Application Publication
(10) Pub. No.: US 2009/0124842 A1
(43) Pub. Date: May 14, 2009

(54) FCC CATALYST FOR LIGHT OLEFIN PRODUCTION

(76) Inventors: William J. Reagan, Pooler, GA (US); Lawrence L. Upson, Barrington, IL (US)

Correspondence Address: HONEYWELL INTERNATIONAL INC;PATENT SERVICES, 101 COLUMBIA DRIVE, P O BOX 2245 MAIL STOP AB/2B, MORRISTOWN, NJ, 07962, US

(21) Appl. No.: 11/456,911

(22) Filed: Jul. 12, 2006

(51) Int. Cl. C07C 4/06; B01J 29/04

(52) U.S. Cl. 585/653; 502/61; 502/71; 502/67; 502/73

(57) ABSTRACT

An improved cracking catalyst is disclosed for the production of propylene from a hydrocarbon feedstock. The process uses a catalyst blend comprising a large pore catalyst and a medium or small pore catalyst, where the medium or small pore catalyst includes a metal deposited on the medium or small pore catalyst.

### FIELD OF THE INVENTION

The present invention relates to a process for the production of light olefins from a hydrocarbon feed stream. This invention also relates to an improved catalyst used in the Fluid Catalytic Cracking process for producing propylene.

### BACKGROUND OF THE INVENTION

Catalytic cracking is the process of breaking larger hydrocarbon molecules into smaller hydrocarbon molecules through contacting the larger hydrocarbon molecules with a catalyst at reaction conditions. The catalytic cracking process is one method used to produce ethylene and propylene from hydrocarbon feedstocks. The ethylene and propylene are important chemicals for the production of the respective plastics polyethylene and polypropylene, two important plastics having a wide variety of uses, such as a material for fabrication of products and as a material for packaging. Other uses of these chemicals include the production of vinyl chloride, ethylene oxide, ethylbenzene and alcohols. Hydrocarbons used as feedstock for light olefin production include natural gas, petroleum liquids, and carbonaceous materials including coal, recycled plastics or any organic material.

Currently, the majority of propylene production is from steam cracking. However, the demand for propylene is growing faster than the ability of steam crackers production to increase propylene. Fluid catalytic cracking (FCC) provides an alternative method of meeting the demand for the production of propylene.

One process for enhancing propylene yield is disclosed in U.S. Pat. No. 4,980,053, where a deep catalytic cracking process is disclosed. The process requires 5-10 seconds of contact time, and uses a mixture of Y-type zeolite and a pentasil, shape-selective zeolite. However, the process reports relatively high yields of dry gas.

Other patents disclose short catalyst contact times, but do not recognize significant light olefin yields, such as in U.S. Pat. No. 5,965,012 which discloses an FCC process, but a better example showing the importance of short contact times is found in U.S. Pat. No. 6,538,169 showing short contact times during the cracking process in the riser reactor. Another FCC process is disclosed in U.S. Pat. No. 6,010,618 where there is a very short catalyst and feed contact time in the riser, and the cracked product is quickly removed below the outlet of the riser. Other patents, such as U.S. Pat. No. 5,296,131 disclose very short FCC catalyst contact times, but these processes are operated to improve gasoline production rather than production of light olefins.

Other patents, U.S. Pat. No. 4,787,967, U.S. Pat. No. 4,871,446, and U.S. Pat. No. 4,990,314, disclose the use of two component catalysts used in FCC processes. The two component catalyst systems use a large-pore catalyst for cracking large hydrocarbon molecules and a small-pore catalyst for cracking smaller hydrocarbon molecules.

To enhance propylene yields, shape selective additives are used in conjunction with conventional FCC catalysts containing Y-zeolites. The additives all have essentially the same selectivity characteristics. The problem with current catalysts is that selectivity is limited, and the amount of propylene produced is only a function of the amount of additive used in the catalyst mixture.

While much research has gone into trying new catalysts for enhancing propylene production, understanding the proper paradigm for selectivity as a function of shape selective catalyst content and additive can increase the propylene yield while operating at a lower temperature, and with a reduced dry gas production and reduced coking. An increased yield can increase the profitability of propylene production, and a small improvement in the catalyst can result in a large improvement in the yields of propylene from hydrocarbon feedstocks.

### SUMMARY OF THE INVENTION

The invention provides a new catalyst blend for increasing propylene yields during a catalytic cracking process. The catalyst blend comprises a first catalyst having a large pore zeolite or molecular sieve blended with a second catalyst molecular sieve having a medium or smaller pore size where the second catalyst has a metal deposited on the catalyst in an amount between about 0.1% and 5% by weight. The catalyst blend comprises the first catalyst in an amount between 20% and 90% by weight, and the second catalyst in an amount between 10% and 80% by weight. The metal is at least one metal selected from the group consisting of gallium, copper, zinc, germanium, cadmium, indium, tin, mercury, thallium and lead, where the total amount of the metal is between 0.1% and 5% by weight.

In another embodiment, the invention comprises contacting a hydrocarbon stream with the above catalyst blend.

Other objects, advantages and applications of the present invention will become apparent to those skilled in the art from the following detailed description.

### DETAILED DESCRIPTION OF THE INVENTION

It has been found that using a small but proper amount of metal and the proper choice of metal or metals on a shape selective small or medium pore size molecular sieve significantly improves the yield of propylene during the catalytic cracking process when blended with a large pore molecular sieve catalyst.

The present invention provides for a catalyst blend, for use in the catalytic cracking process. The catalyst blend comprises a first catalyst comprising a large pore molecular sieve in an amount between about 20% and 90% by weight, and a second catalyst comprising a medium or small pore size molecular sieve in an amount between about 10% and 80% by weight, and where the second catalyst has a metal deposited on the catalyst in an amount from about 0.1% to about 5% by weight of the second catalyst.

Each of the catalysts comprise a molecular sieve with an inorganic oxide binder, a filler, or both to provide the desired level of mechanical strength and attrition resistance of the bound catalyst. The amount of binder and/or filler material contributes from about 20% to about 80% of the total catalyst weight. In addition to enhancing the catalyst strength properties, the binder and/or filler materials allow the molecular sieve to be bound into larger particle sizes suitable for commercial catalytic purposes. Binders and fillers are known in the art and not enumerated here. Examples of binders and fillers are described in U.S. Pat. No. 6,649,802 which is incorporated by reference in its entirety. The term catalyst as used in this application refers to the molecular sieve with the binder and/or filler in a state useable for commercial catalytic purposes. The catalyst blend, when used herein, refers to a mixture of the first and second catalysts, and can be a physical mixture, or a blend that combines both catalysts into a single catalyst particle.

The metal, or metals, deposited on the second catalyst are deposited after the catalyst is formed with the binder and/or filler, and can be dispersed within the molecular sieve, or deposited on the external surface of the catalyst particles, or some combination.

The catalyst is a shape selective zeolite or molecular sieve for use in cracking larger hydrocarbon molecules to propylene. Large pore molecular sieves are molecular sieves with pore opening diameters greater than about 0.7 nm, and are typically defined by 12 membered or larger rings. A typical first catalyst is a Y-zeolite. Large pore Y-zeolites are known in the art and include H-Y, RE-Y, US-Y, NH4-Y, and LZ-210, which are described in U.S. Pat. Nos. 4,842,836, 4,965,233, 6,616,899, and 6,869,521 and which are incorporated by reference in their entirety. Molecular sieves with medium and small pore sizes are characterized as having effective pore opening diameters of less than or equal to about 0.7 nm, and with the pore ring sizes having 10 or fewer members. One such molecular sieve has an MFI type structure, and preferably is ZSM-5 or ST-5 molecular sieve. Other zeolites useable for the second catalyst in the present invention include, but are not limited to, ZSM-11, ZSM-22, Beta, erionite, ZSM-34 and SAPO-11. In one embodiment, the metal is selected from gallium, copper, zinc and mixtures thereof, where the total metal content is between 0.1% and 5% by weight of the second catalyst, and preferably between 0.5% and 2% by weight of the second catalyst.

The preferred second catalyst is a zeolite and has an MFI type structure and the preparation is known in the art as exemplified by U.S. Pat. No. 5,254,327 and is incorporated by reference in its entirety. However, the metal was deposited on the catalyst by the standard “incipient wetness” technique. The metals in the form of soluble metal salts, were dissolved in sufficient water to fill the pore volume of the catalyst. The metal solution was then added, dropwise, to the catalyst powder while stirring the catalyst in water. After depositing the metal in solution, the catalyst is dried at about 93° C. (200° F.) and calcined at about 540° C. (1000° F.). Other methods of depositing the metals on the catalyst include ion exchange methods, or even incorporation of the metals during the zeolite synthesis step.

In another embodiment, the second catalyst comprises a shape selective zeolite or molecular sieve having at least two metals deposited on the catalyst, where the total amount of the metals deposited is from about 0.1% to about 5% by weight. The metals deposited are selected from gallium, copper, zinc, germanium, cadmium, indium, tin, mercury, thallium, lead and mixtures thereof. Preferably, the metals are selected from gallium, copper and zinc, and the metals are each deposited in an amount between 0.1% and 2% by weight. For this embodiment, when selecting two metals and depositing the metals on the catalyst, it is preferred that the metals are deposited in substantially equal amounts by weight.

The process of the present invention is described in the context of an FCC process. An FCC arrangement consists of a riser reactor that provides a pneumatic conveyance zone in which the reaction takes place, a separation unit in which the product gas leaving the riser reactor is separated from the catalyst blend, a regenerator that receives the catalyst blend and regenerates it (via coke combustion with air or suitable oxygen mixture) for reuse, and a blending vessel that mixes the catalyst blend with a fluidizing gas prior to feeding the catalyst blend and a hydrocarbon feedstream into the riser reactor. FCC technology is known, as shown in U.S. Pat. No. 6,538,169, and is incorporated by reference in its entirety.

The catalyst blend, as described above, is mixed with a fluidizing gas, and fed with a hydrocarbon feedstream into the riser reactor, where the catalyst blend and hydrocarbon gas react under reaction conditions to generate a product gas that includes propylene.

Hydrocarbon feedstocks suitable for processing in this invention include, but are not limited to, naphthas having a boiling range above 50° C. (122° F.), vacuum gas oil having a boiling range from 343° C. to 552° C. (650° F. to 1025° F.) and is prepared by vacuum fractionation of atmospheric residue, and heavy or residual feeds having boiling ranges above 499° C. (930° F.).

The riser typically operates with dilute phase conditions above the point of feed injection wherein the density is usually less than 320 kg/m³ (20 lb/ft³) and, more typically, less than 160 kg/m³ (10 lb/ft³). The feedstream will ordinarily have been heated to a temperature in a range of from 150° C. to 320° C. (300° F. to 600° F.), before contacting the catalyst. Additional amounts of feed may be added downstream of the initial feed point.

In an effort to minimize the contact time of the feed and the catalyst blend which may promote further conversion of desired products to undesirable other products, the catalyst blend is rapidly separated from the product gas. Contact times in the riser reactor are from 0.8 seconds to 3.5 seconds. A variety of separation means are known in the art and are not detailed here.

The catalyst blend to hydrocarbon feed ratio by weight is in the range from about 5 to 50 and preferably from about 5 to 30, and more preferably from 5 to 15.

Low hydrocarbon partial pressure operates to favor the production of light olefins. Accordingly, the riser pressure is set at about 140 to 420 kPa (20 to 60 psia) with a hydrocarbon partial pressure of about 35 to 310 kPa (5 to 45 psia), with a preferred hydrocarbon partial pressure of about 70 to 140 kPa (10 to 20 psia). This relatively low partial pressure for hydrocarbon is achieved by using steam as a diluent to the extent that the diluent is 2-40 wt-% of feed and preferably about 10-20 wt-% of feed. Other diluents such as dry gas can be used to reach equivalent hydrocarbon partial pressures.

The temperature of the cracked stream at the riser outlet will be about 510° C. to 621° C. (950° F. to 1150° F.). However, we have found that riser outlet temperatures above 566° C. (1050° F.) make more dry gas and little more olefins so the preferred temperature is from about 510° C. to 566° C. (950° F. to 1050° F.).

### EXAMPLE

Testing was performed in an ACE™ testing microreactor unit. ACE units are available from Xytel Corp. in Elk Grove Village, Ill. The hydrocarbon feedstream was a light naphtha and the reaction conditions included a temperature of 565° C. (1050° F.) and a catalyst to hydrocarbon ratio of about 5 over the catalyst.

The test results from the ACE unit are summarized in Table 1. The comparisons are with a commercial catalyst having a ST-5 molecular sieve in an amount of approximately 25% by weight and the molecular sieve with different amounts of activated ST-5s with metals deposited on the catalyst. ST-5 is an MFI type zeolite and is disclosed in U.S. Pat. No. 5,254,327 which is incorporated by reference in its entirety.

TABLE 1

| | | 1% Ga on | 1% Zn on | Zn—Cu on | 1% Ni on |
|---|---|---|---|---|---|
| Selectivities | ST-5 | ST-5 | ST-5 | ST-5 | ST-5 |
| H2 | 0.11 | 0.27 | 0.81 | 0.40 | 0.55 |
| C1 | 0.55 | 0.80 | 1.65 | 0.80 | 2.10 |
| C2 | 1.30 | 1.70 | 1.35 | 1.40 | 2.20 |
| C2= | 3.15 | 4.50 | 3.70 | 3.70 | 3.70 |
| C3= | 6.0 | 7.30 | 6.40 | 6.90 | 5.50 |
| C3 | 4.75 | 5.20 | 3.85 | 4.10 | 3.85 |
| nC4 | 1.53 | 1.70 | 1.70 | 1.53 | 1.47 |
| iC4 | 0.75 | 0.90 | 1.80 | 0.90 | 0.60 |
| iC4= | 1.55 | 1.64 | 1.55 | 1.55 | 1.25 |
| nC4= | 2.15 | 2.25 | 2.15 | 2.15 | 1.75 |
| C4== | <0.01 | 0.035 | 0.033 | 0.035 | 0.026 |
| C5+ | 77.36 | 69.70 | about 75 | about 76 | 70.40 |
| Coke | 0.8 | 4 (est.) | Unknown | Unknown | 6.6 |

The results show that a small amount of additive to the ST-5 catalyst generated an increased amount of propylene produced from a naphtha feedstock, over the commercial ST-5 catalyst. Metals that showed an increase were gallium with a 21.7% increase, zinc with a 6.7% increase, and a 50-50 mixture of zinc and copper showed a 15% increase. On the other hand, improper choice of additive can result in degradation of propylene production. Other possible combinations from the present results indicate a mixture of gallium and copper, and a mixture of gallium and zinc.

By limiting the amounts of additives, and by selecting the proper metals to add to the catalysts, propylene production can be substantially increased.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A catalyst blend for fluidized catalytic cracking to increase propylene production, comprising:
a first catalyst comprising a large pore molecular sieve in an amount from about 20% to about 90% of the catalyst in the catalyst blend by weight;
a second catalyst comprising a medium or small pore molecular sieve in an amount from about 10% to about 80% of the catalyst blend by weight;
wherein the second catalyst comprises a metal deposited on the second catalyst in an amount from about 0.1% to about 5% by weight of the second catalyst.

2. The catalyst blend of claim 1 wherein the metal is selected from the group consisting of gallium, copper, zinc, indium, cadmium and mixtures thereof.

3. The catalyst blend of claim 1 wherein the metal is present in an amount from about 0.5% to about 2% of the catalyst by weight.

4. The catalyst blend of claim 1 wherein the metal comprises at least two metals selected from the group consisting of gallium, copper, zinc, germanium, cadmium, indium, tin, mercury, thallium and lead.

5. The catalyst blend of claim 4 wherein the metal comprises at least two metals selected from the group consisting of gallium, copper and zinc.

6. The catalyst blend of claim 4 wherein each metal comprises an amount between 0.1% and 2% of the second catalyst by weight.

7. The catalyst blend of claim 4 wherein the metals are present in substantially equal weight amounts.

8. The catalyst blend of claim 1 wherein the second catalyst molecular sieve has an MFI type structure.

9. The catalyst blend of claim 8 wherein the second catalyst molecular sieve is ZSM-5 or ST-5.

10. The catalyst blend of claim 1 wherein the second catalyst molecular sieve is selected from the group consisting of ZSM-5, ZSM-11, ZSM-22, beta, erionite, ZSM-34, SAPO-11, ST-5, and mixtures thereof.

11. The catalyst blend of claim 1 wherein the first catalyst is a Y-zeolite.

12. The catalyst blend of claim 11 wherein the first catalyst is selected from the group consisting of H-Y, NH4-Y, RE-Y, US-Y, LZ-210, and mixtures thereof.

13. A process for propylene production, comprising:
contacting a hydrocarbon feedstream with a catalyst blend of a first catalyst and a second catalyst, wherein the first catalyst comprises a large pore molecular sieve in an amount from about 20% to about 90% by weight and the second catalyst comprises a medium or small pore size molecular sieve in an amount from about 10% to about 80% by weight and the second catalyst comprises a metal deposited on the second catalyst, at reaction conditions thereby producing an effluent stream comprising propylene; and
separating the effluent stream into an enriched propylene stream and a second stream comprising cracked hydrocarbon products.

14. The process of claim 13 wherein the metal deposited on the second catalyst is selected from the group consisting of gallium, copper, zinc, germanium, cadmium, indium, tin, mercury, thallium, lead, and combinations thereof.

15. The process of claim 14 wherein the metal deposited on the second catalyst comprises at least two metals selected from the group consisting of gallium, copper, zinc, germanium, cadmium, indium, tin, mercury, thallium, lead, and combinations thereof.

16. The process of claim 13 wherein the reaction conditions comprise a temperature between about 900° C. and about 1100° C.

17. The process of claim 13 wherein the reaction conditions comprise a catalyst to hydrocarbon ratio by weight from about 5 to about 30.

18. The process of claim 17 wherein the reaction conditions comprise a catalyst to hydrocarbon ratio by weight from about 5 to about 15.

19. The process of claim 13 wherein the reaction conditions comprise a pressure between about 140 kPa (20 psia) and about 420 kPa (60 psia).
